(19) United States
(12) Patent Application Publication
(10) Pub. No.: US 2024/0342069 A1
(43) Pub. Date: Oct. 17, 2024

(54) Composition Comprising Fatty Acid Amidoalkyl Betaine and Carboxymethylated Derivative of a Dimethylaminoalkylamine

(71) Applicant: Kensing LLC, Kankakee, IL (US)

(72) Inventors: Dominik SCHUCH, Düsseldorf (DE); Uwe BEGOIHN, Essen (DE); Christian HARTUNG, Essen (DE); Hans Henning WENK, Mülheim an der Ruhr (DE); Ralf KLEIN, Velbert (DE)

(21) Appl. No.: 18/682,512

(22) PCT Filed: Mar. 31, 2022
(86) PCT No.: PCT/EP2022/058649

§ 371 Date: Feb. 9, 2024

(30) Foreign Application Priority Data
Aug. 30, 2021 (EP) 21193761.0

(51) Int. Cl. A61K 8/44; A61K 8/36; A61Q 19/10

(52) U.S. Cl. CPC A61K 8/442; A61K 8/361; A61Q 19/10

(57) ABSTRACT

The present invention provides a composition comprising at least one fatty acid amidoalkyl betaine and at least one derivative of a dimethylaminoalkylamine and also a process for producing a surfactant.

### FIELD OF THE INVENTION

The present invention provides a composition comprising at least one fatty acid amidoalkyl betaine and at least one derivative of a dimethylaminoalkylamine and also a process for producing a surfactant.

### PRIOR ART

In the prior art, concentrated fatty acid amidoalkyl betaines are produced in the presence of certain fluidizing agents such as beet betaine, fatty acids or ethoxylated glycerides, since the viscosity of the reaction mixture in the betaine synthesis, known as the gel phase, otherwise increases to the extent that simple handling is no longer possible.

Thus, DE2926479 describes a process for preparing betaines based on fatty acids having 6 to 18 carbon atoms by quaternization of fatty acid amides with ω-haloalkylcarboxylic acids in aqueous solution.

EP 1 659 109 discloses a process for preparing highly concentrated flowable and pumpable aqueous solutions of betaines having a betaine content of at least 32% by weight by quaternization of compounds containing tertiary amine nitrogen with ω-halocarboxylic acids with addition of micellar thickeners.

EP 0 560 114 discloses aqueous liquid solutions of a betaine based on acyl radicals of preferably hardened coconut oil fatty acids or a fatty acid mixture corresponding on average to coconut oil fatty acid, the solution having a solids content of at least 40% by weight, a pH of 5 to 8 and an aminoamide content of ≤1% by weight, characterized by a content of 1% to 3% by weight (based on solution) of one or more saturated fatty acids having on average 8 to 18 carbon atoms or one or more unsaturated fatty acids having on average 8 to 24 carbon atoms and 0% to 4% by weight (based on solution) of glycerol.

The object of the present invention was to make it possible to obtain betaine-containing compositions of low viscosity.

### DESCRIPTION OF THE INVENTION

It has surprisingly been found that the presence of dimethylaminoalkylamine in the reaction mixture of fatty acid amidoalkyl dimethylamines and chloroacetic acid yields fatty acid amidoalkyl betaines in a mixture with carboxymethylated dimethylaminoalkylamine derivatives that, both during the synthesis and in the final product too, exhibit a lower viscosity than the products known from the prior art.

The present invention therefore provides compositions comprising at least one fatty acid amidoalkyl betaine and at least one derivative of a dimethylaminoalkylamine according to claim 1. The invention further provides formulations comprising compositions according to the invention according to claim 8.

The invention further provides a process for producing a betaine-containing composition according to claim 10.

An advantage of the composition according to the invention is that the production of more concentrated products is made easier by a less pronounced or completely avoided gel phase.

A further advantage of the composition according to the invention is that products of higher concentration are obtainable.

A further advantage is that the addition of preservatives can be eliminated or is necessary only to a limited extent.

A further advantage of the composition according to the invention is that the viscosity of the end products is reduced compared to products of the prior art with the same concentration.

A further advantage of the composition according to the invention is that the color stability at elevated temperatures is increased in comparison to non-inventive compositions.

A further advantage of the composition according to the invention is that the viscosity of cleansing formulations comprising the inventive compositions including additional fatty acid is increased.

A further advantage of the composition according to the invention is that the skin feel during washing and the foam creaminess of cleansing formulations comprising the inventive compositions is increased.

The present invention thus provides a composition comprising
A) at least one fatty acid amidoalkyl betaine of general formula I)

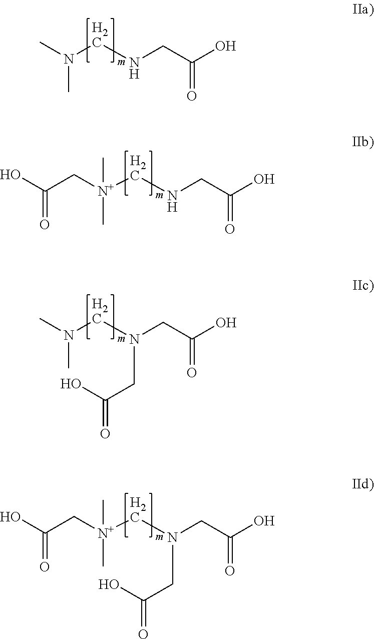

where
n=1 to 10, preferably 2 to 5, in particular 3, and
R¹CO=acyl radical of a fatty acid,
B) at least one derivative of a dimethylaminoalkylamine selected from the group

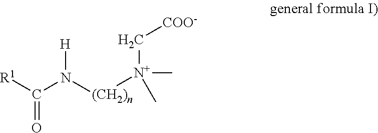

where
m=1 to 10, preferably 2 to 5, in particular 3.

Unless otherwise stated, all stated percentages (%) are percentages by weight.

Fatty acid amidoalkyl betaines of general formula I) are quantified by those skilled in the art in accordance with the article “Titrimetric methods for the determination of betains” published in the Application Bulletin of Metrohm AG, No. 264/1 d.

The proportion by mass of component B) is in the context of the present invention determined by HPLC as the sum of the four derivatives IIa) to IId).

For this, 100 mg of the composition under investigation are dissolved in 10 ml of a solution consisting of water and ethanol (20:80). An aliquot of the solution is analysed by HPLC. The analysis is carried out using an HPLC system equipped as follows:
Instrument: LC Agilent AG1260
Column: TSK Gel Amide-80 from TOSH, 2.0*150 mm, 3 μ
Oven temperature: 30° C.
Eluent: A: 0.04% by volume H₂SO₄ in water
   B: Acetonitrile
Injected volume: 20 μl
Detector: DAD 210 nm
Flow: 0.5 ml/min

### Gradient:

| Time [min] | Eluent B [%] |
|---|---|
| 0 | 95 |
| 2 | 95 |
| 20 | 40 |
| 25 | 40 |
| 30 | 95 |
| 40 | 95 |

Analysis time: 40 min.

5 Component B) is separated from the matrix based on its different polarity compared to component A).

For evaluation, an external calibration is carried out using a standard solution of an industrially produced component B) in the form of a mixture of derivatives composed of 25% each of IIa) to IId), and a calibration line is generated. Through evaluation of the peak areas, it is then possible to determine the concentration of the analytes in the sample.

Preferred compositions in accordance with the invention comprise a component A) in which n and m are identical, in particular where n=m=3.

Preferred R¹CO in accordance with the invention are acyl radicals selected from the group of acyl radicals of natural fatty acids.

Preferred fatty acids in this connection are mixtures of natural fatty acids, in particular vegetable fatty acids, more preferably fatty acids from coconut oil or palm kernel oil, in particular mixtures in which no carboxylic acid chain length has a proportion in the overall chain length distribution of more than 95% by weight, in particular more than 70% by weight.

From this it follows that preferred compositions in accordance with the invention comprise a plurality of fatty acid amidoalkyl betaines of general formula I) having different radicals R¹.

Natural fatty acids can be produced based on naturally occurring vegetable or animal oils and have preferably 6 to 30 carbon atoms, in particular 8 to 22 carbon atoms. Natural fatty acids are generally unbranched and usually consist of an even number of carbon atoms. Any double bonds have cis configuration. Examples are: caproic acid, caprylic acid, capric acid, lauric acid, myristic acid, palmitic acid, palmitoleic acid, pelargonic acid (obtainable for example from the ozonolysis or oxidative cleavage of oleic acid), isostearic acid, stearic acid, 12-hydroxystearic acid, dihydroxystearic acid, undecylenic acid (obtainable from the pyrolysis of ricinoleic acid), oleic acid, linoleic acid, linolenic acid, petroselinic acid, elaidic acid, arachic acid, behenic acid, erucic acid, gadoleic acid, eicosapentaenoic acid, docosahexaenoic acid and arachidonic acid.

Particularly preferably in accordance with the invention, the acyl group donor is selected from fatty acid acyl group donors that are characterized in that they provide an acyl group mixture comprising at least two acyl groups of the carboxylic acids selected from the group of caproic acid, caprylic acid, capric acid, lauric acid, myristic acid, palmitic acid, palmitoleic acid, pelargonic acid (obtainable for example from the ozonolysis or oxidative cleavage of oleic acid), isostearic acid, stearic acid, 12-hydroxystearic acid, dihydroxystearic acid, undecylenic acid (obtainable for example from the pyrolysis of ricinoleic acid), oleic acid, linoleic acid, linolenic acid, petroselic acid, elaidic acid, arachic acid, behenic acid, erucic acid, gadoleic acid, eicosapentaenoic acid, docosahexaenoic acid and arachidonic acid.

The composition of the present invention is preferably an aqueous composition. The term “aqueous composition” is understood to mean that it contains water, more particularly in an amount of from 0.01% by weight to 95.0% by weight, preferably from 40.0% by weight to 75.0% by weight, more preferably from 45.0% by weight to 60.0% by weight, the percentages by weight being based on the total composition.

It is preferable in accordance with the invention when an aqueous composition of the invention has a pH within a range of from 3 to 13, preferably 3 to 7 and 10 to 13, more preferably 4.0 to 6.5. The “pH” in connection with the present invention is defined as the value measured using a pH electrode calibrated to ISO 4319 (1977), without diluting the mixture, at 25° C. (unless a different temperature is specified) after stirring for 5 minutes.

It is obvious that components A) and B) comprise structures that are protonated or deprotonated, for example on the amine and/or carboxyl groups, depending on the pH present in the composition.

A preferred composition according to the invention is characterized in that n=m=3.

In accordance with the invention, the derivatives IIa) to IId) are protonated or deprotonated on the carboxymethyl radical.

A preferred composition according to the invention is characterized in that component B) is present in an amount of from 0.25% by weight to 10.0% by weight, preferably from 0.35% by weight to 7.0% by weight, more preferably from 0.5% by weight to 4.0% by weight, the percentages by weight being based on the total composition.

The composition of the present invention preferably comprises at least two, preferably all, of derivatives IIa) to IId).

It is preferable in accordance with the invention when the weight ratio of component A) to component B) in the composition according to the invention is in the range from 125:1 to 5:1, preferably from 100:1 to 7:1, more preferably from 75:1 to 9:1.

A preferred composition according to the invention is characterized in that it comprises
C) at least one free fatty acid or salt thereof.

In this connection, the preferred fatty acids already mentioned above in connection with R¹ are preferably present.

The fatty acid content can be determined by HPLC analysis according to M. J. Cooper, M. W. Anders. Anal. Chem., 1974, 46 (12), pp. 1849-1852.

It is preferable in accordance with the invention that component C) is present in the composition according to the invention in an amount of from 0.1% by weight to 6.0% by weight, preferably from 0.3% by weight to 4.5% by weight, more preferably from 0.5% by weight to 3.0% by weight, the percentages by weight being based on the total composition.

A preferred composition according to the invention is characterized in that the fatty acid of component C) is identical to R¹COOH with the R¹ of general formula I).

In addition, the typical by-products and impurities of industrial betaine mixtures known to those skilled in the art may be present in the composition according to the invention. These include for example sodium chloride, glycerol, fatty acid amidoalkyl dimethylamines and associated ammonium salts, glycolic acid and salts thereof, citric acid and citrates as pH adjusters and small amounts of <100 ppm each of monochloroacetic acid and dichloroacetic acid and salts thereof.

It is particularly preferable in accordance with the invention that the composition comprises 3.0% by weight to 10% by weight of sodium chloride and 1.0% by weight to 5.0% by weight of glycerol.

The invention further provides a process for producing a betaine-containing composition, more particularly a composition according to the invention, comprising the process steps of
a) providing at least one fatty acid amidoalkyl dimethylamine, in particular a fatty acid amidopropyl dimethylamine, and at least one dimethylaminoalkylamine, in particular dimethylaminopropylamine,
b) reacting the provided fatty acid amidoalkyl dimethylamine and dimethylaminoalkylamine with chloroacetic acid to form fatty acid amidoalkyl betaine and at least one at least singly carboxymethylated dimethylaminoalkylamine.

It is obvious that the provided fatty acid amidoalkyl dimethylamine and dimethylaminoalkylamine, the chloroacetic acid and the at least singly carboxymethylated dimethylaminoalkylamine may also be present at least partly in the respective protonated or deprotonated salt form, depending on the pH present in the process step.

It is preferable in accordance with the invention when process step b) of the process according to the invention is carried out at a temperature within a range from 60° C. to 120° C., more preferably 80° C. to 100° C.

A process that is preferred in accordance with the invention is characterized in that process step b) of the process according to the invention is carried out in the presence of water, more preferably in the presence of water and glycerol.

It is possible to carry out process step b) of the process according to the invention within a broad pH range. It is preferable in accordance with the invention when the reaction in process step b) of the process according to the invention is carried out within a pH range from 7.5 to 10.5, preferably from 8.0 to 10.0, measured undiluted at 98° C.

A process that is preferred in accordance with the invention is characterized in that, in process step b) of the process according to the invention, the pH, more particularly the pH range from 7.5 to 10.5, preferably from 8.0 to 10.0, measured undiluted at 98° C., is adjusted by addition of an alkali metal hydroxide, more particularly NaOH, preferably in the form of an aqueous solution.

A process that is preferred in accordance with the invention is characterized in that the weight ratio of the fatty acid amidoalkyl dimethylamines, in particular fatty acid amidopropyl dimethylamines, provided in process step a) to the dimethylaminoalkylamines, in particular dimethylaminopropylamine, provided in process step a) is in the range from 99.7:0.3 to 90:10, preferably from 99.6:0.4 to 95:5, more preferably from 99.5:0.5 to 98:2.

The fatty acid amidoalkyl dimethylamine content is determined by those skilled in the art by HPLC. For this, the fatty acid amidoalkyl dimethylamines are separated from the sample matrix through ion-exchange HPLC and quantified by UV detection with the aid of external single-point calibration.

The dimethylaminoalkylamine content is determined by those skilled in the art by HPLC with a UV detector after derivatization of the respective amine with phenyl isothiocyanate (PITC) to the phenylurea derivative. The derivatization is the first step in the reaction of the phenylthiohydantoin method (Edman, 1950). The analyses are carried out with a high-purity RP 18 column with gradient program and the UV detection is carried out at 254 nm. Quantitative data are determined with the aid of an external calibration standard.

A process that is preferred in accordance with the invention is characterized in that it includes a process step c) in which the pH of the betaine-containing composition from process step b) is adjusted with an organic or an inorganic acid to pH 3 to 7, preferably pH 4.0 to 6.5.

The compositions according to the invention can advantageously be used for the production of aqueous formulations, preferably care and cleaning formulations, in particular cosmetic and dermatological formulations. The present invention accordingly likewise provides formulations, in particular cosmetic and dermatological formulations, comprising the compositions according to the invention.

The present invention also provides for the use of the compositions of the invention for the production of corresponding formulations.

Compositions preferred in accordance with the invention, more particularly formulations, additionally include at least one further surfactant, in particular sodium laureth sulfate. Such preferred formulations include for example shampoos, shower gels, bath oils, liquid soaps, mouthwashes, household cleaners, industrial cleaners, dishwashing detergents, textile care compositions and textile finishing compositions.

The following examples describe the present invention by way of example, without any intention to limit the invention, the scope of application of which is apparent from the entirety of the description and the claims to the embodiments specified in the examples.

### EXAMPLES

### Example 1: Preparation of a Composition According to the Invention

A mixture of 148.2 g of coconut oil fatty acid amidopropyldimethylamine made from hydrogenated coconut oil having a typical mass distribution of acyl radicals (based on acyl radicals present having 8 to 18 carbon atoms: C8: 5-8%; C10: 5-8%; C12: 45-49%; C14: 16-20%; C16: 8-11%; C18 (total): 10-14%; less than 2% of unsaturated acyl radicals) and 1.8 g of dimethylaminopropylamine (DMAPA), which had an amine value of 196 mg KOH/g (0.53 mol, based on molar mass of mixture calculated from the amine value) was slowly added to a mixture of 205.5 g of water, 63.4 g of 80% aq. monochloroacetic acid (0.54 mol) and 43.0 g of a 50% aq. sodium hydroxide solution (0.54 mol) at 22° C. under stirring,

The reaction mixture was then heated to 98° C. while stirring and the pH of the undiluted mixture was constantly kept at 8-9 with a further 7.5 g of 50% aq. sodium hydroxide solution. Once the residual content of coconut oil fatty acid amidopropyldimethylamine had reached a value of <0.3% by weight based on the total composition, the pH of the undiluted composition was adjusted to 4.6 with 3.2 g of a 50% aq. citric acid solution and the mixture was allowed to cool to 22° C. The reaction product was a yellowish, clear and thin liquid. It contained 39% by weight of component A, 2.1% by weight of component B and 6.5% by weight of sodium chloride.

### Example 2: Preparation of a Non-Inventive Composition

150 g of coconut oil fatty acid amidopropyldimethylamine from hydrogenated coconut oil having a typical mass distribution of acyl radicals (based on acyl radicals present having 8 to 18 carbon atoms: C8: 5-8%; C10: 5-8%; C12: 45-49%; C14: 16-20%; C16: 8-11%; C18 (total): 10-14%; less than 2% of unsaturated acyl radicals) (0.44 mol, calculated from the amine value) were slowly added to a mixture of 200 g of water, 53.4 g of 80% aq. monochloroacetic acid (0.45 mol) and 36.2 g of a 50% aq. sodium hydroxide solution (0.45 mol) at 22° C. under stirring, The reaction mixture was then heated to 98° C. while stirring and the pH of the undiluted mixture was constantly kept at 8-9 with a further 6.4 g of 50% aq. sodium hydroxide solution. For reducing the residual content of coconut oil fatty acid amidopropyldimethylamine to a value of <0.3% by weight based on the total composition, it was necessary to add a further 2.5 g of 80% aq. monochloroacetic acid solution (0.02 mol). Once the target value was reached, the pH of the undiluted composition was adjusted to 4.6 with 3.5 g of a 50% aq. citric acid solution and the mixture was allowed to cool to 22° C. The reaction product was a yellowish solid gel at 22° C. that was neither flowable nor stirrable. It contained 37% by weight of component A, 0.13% by weight of component B and 6.3% by weight of sodium chloride.

### Example 3: Preparation of a Non-Inventive Composition According to Example 12 of EP0656346

In example 12 of EP0656346, 17.8 g of the product made in example 11 of EP0656346, consisting of a calculated amount of 24.5% by weight of carboxylated DMAPA (calculation based on an average molecular weight of 218 g/mol), were added to yield 4.36 g of component B in the final product.

Simultaneously, 320 g of the product made in example 1 of EP0656346 consisting of 290 g of coconut oil fatty acid amidopropyldimethylamine (and 30 g glycerol) are reacted in example 12 of EP0656346 to yield 347 g of component A).

With the total composition having a mass of 1033.5 g, this results in a calculated concentration of 33.6% by weight of component A and 0.42% by weight of component B.

### Example 4: Preparation of A Non-Inventive Composition According to Example 12 or EP0656346 with an Increased Product Concentration

Example 12 of EP0656346 was repeated with the coconut oil fatty acid amidopropyldimethylamine from example 1 of EP0656346 and a decreased amount of water to yield a final product containing 38% of component A, which was a highly viscous gel.

### Example 5: Exothermic Reaction

The reaction mixtures described in the above examples 1, 2 and 4 showed an exothermic behaviour when a temperature of 70° C. is reached by external heating. This is very relevant with regard to plant security, since an industrial scale production of such betaines is typically done in batch sizes of 20 to 80 tons.

The exothermic behaviour was less pronounced during the synthesis described in example 1. After reaching 70° C. by heating, the temperature increased from 70 to 80° C. within 15 minutes without additional heating. In contrast to this mild exothermic behaviour, the temperature increased from 70° C. to even 85° C. in just 5 minutes without additional heating during the syntheses described in the examples 2 and 4.

This unexpected behaviour of the reaction in example 1 is a clear advantage in the controllability of the process.

### Example 6: Preparation of a Composition According to the Invention

6.0 g coconut oil fatty acid having a typical mass distribution of acyl radicals (based on acyl radicals present having 8 to 18 carbon atoms: C8: 5-8%; C10: 5-8%; C12: 45-49%; C14: 16-20%; C16: 8-11%; C18 (total): 10-14%; less than 2% of unsaturated acyl radicals) were added to the final product described in example 1 and the mixture was stirred for 2 h at 90° C. to yield a yellowish, clear and thin liquid with a fatty acid content of 1.7% by weight.

In example 7 and 9 the ingredients are named according to the International Nomenclature of Cosmetic Ingredients, abbreviated INCI. These are unique identifiers for cosmetic ingredients that are assigned in accordance with rules established by the Personal Care Products Council (PCPC).

### Example 7: Color Stability of Formulations Containing Betaines

Aqueous cleansing formulations containing 9% by weight Sodium Laureth Sulfate (28% by weight Texapon® NSO-IS by BASF as an aqueous solution containing 32% by weight Sodium Laureth Sulfate), 3% by weight Cocamidopropyl Betaine of examples 1, 4 and 5 calculated based on component A, 0.5% by weight Sodium Chloride and 2.1% by weight of PEG-18 Glyceryl Oleate/Cocoate (2.5% by weight ANTIL® 171 by Evonik of which 0.4% were water) were analyzed for color stability after 4 weeks of storage at 50° C.

Directly after preparation, all three formulations containing the products made in the examples 1, 4 and 5 showed a similar color of 42 vs. 43 vs. 41 Hazen. After 4 weeks of storage at 50° C., a color of 49 Hazen was measured for the formulation based on example 1, 59 Hazen for the formulation based on example 4 and 47 Hazen for the formulation based on example 5. All color values were determined with a LICO 690 by Hach Lange with a 11 mm round cuvette at 22° C., calibrated against distilled water.

### Example 8: Foaming Properties

The product of example 5, which contained 1.7% by weight coconut oil fatty acid, yielded less foam in SITA measurements of a 0.5% by weight aq. solution after 20 sec. of stirring at 30° C. (water: ˜10° dH, PH ˜6, 1500 rpm) than the product of example 4.

With the product of example 4, the foam volume was analyzed to be 830 ml. With the product ofexample 5, the foam volume was measured to be only 520 ml. This is advantageous in various applications, in which strong foaming are annoying, such as laundry, automated dishwashing, automated car washing, make-up removal or brushing of teeth.

### Example 9: Formulation Viscosity, Skin Feel and Foam Creaminess

The aqueous cleansing formulations described in example 7 (before the storage at 50° C.) showed different viscosities. The viscosity of the formulation based on example 5, which contained additional coconut oil fatty acid, was analyzed to be 3120 mPa s and the viscosity of the formulation based on example 4 was measured to be 2440 mPa s. These results were obtained at 25° C. by a Brookfield viscometer with spindle 62 at 30 rpm.

For comparing the skin care benefits of the products described in examples 4 and 5 in an aqueous cleansing formulation, a sensory hand washing test was carried out according to the state of the art with the respective formulations described in example 7 (before the storage at 50° C.). A group consisting of 10 trained testers washed their hands in a defined manner and evaluated the skin feel and foam creaminess based on a rating scale from 1 (poor) to 5 (very good).

As a result of this test, the formulation based on example 5 received an average panel judgement of 3.8 for the parameter skin feel during washing and 3.9 for the parameter foam creaminess. In contrast, the formulation based on example 4 only received average panel judgements of 3.4 for the parameter skin feel during washing and 3.7 for the parameter foam creaminess.

1. Composition comprising
A) at least one fatty acid amidoalkyl betaine of general formula I) 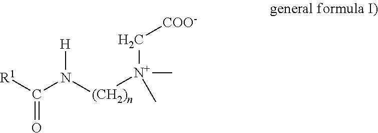
where
n=1 to 10, preferably 2 to 5, in particular 3, and
R¹CO=acyl radical of a fatty acid,
B) at least one derivative of a dimethylaminoalkylamine selected from the group 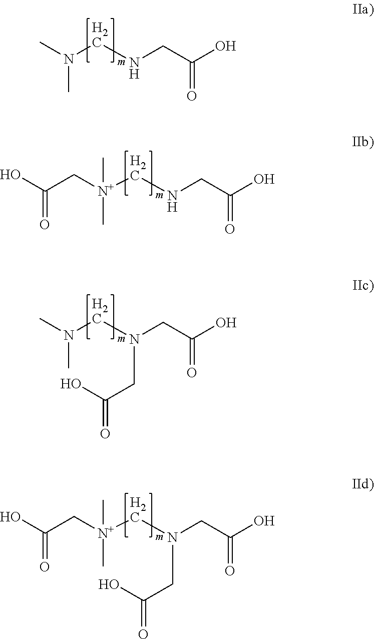
where
m=1 to 10, preferably 2 to 5, in particular 3.

2. Composition according to claim 1, characterized in that the weight ratio of component A) to component B) is in the range from 125:1 to 5:1, preferably from 100:1 to 7:1, more preferably from 75:1 to 9:1.

3. Composition according to claim 1, characterized in that component B) is present in an amount of from 0.25% by weight to 10.0% by weight, preferably from 0.35% by weight to 7.0% by weight, more preferably from 0.5% by weight to 4.0% by weight, the percentages by weight being based on the total composition.

4. Composition according to claim 1,
characterized in that it comprises
C) at least one free fatty acid or salt thereof.

5. Composition according to claim 4, characterized in that component C) is present in an amount of from 0.1% by weight to 6.0% by weight, preferably from 0.3% by weight to 4.5% by weight, more preferably from 0.5% by weight to 3.0% by weight, the percentages by weight being based on the total composition.

6. Composition according to claim 4, characterized in that the fatty acid of component C) is identical to R¹COOH with the R¹ of general formula I).

7. Composition according to claim 4 comprising at least one further surfactant.

8. A care and cleaning formulation comprising the composition of claim 1 and at least one surfactant.

9. A method of producing a care and cleaning formulation comprising providing the composition of claim 1 and at least one surfactant and mixing to form a care and cleaning formulation.

10. Process for producing a betaine-containing composition, comprising the process steps of
a) providing at least one fatty acid amidoalkyl dimethylamine, in particular a fatty acid amidopropyl dimethylamine, and at least one dimethylaminoalkylamine, in particular dimethylaminopropylamine,
b) reacting the provided fatty acid amidoalkyl dimethylamine and dimethylaminoalkylamine with chloroacetic acid to form fatty acid amidoalkyl betaine and at least one at least singly carboxymethylated dimethylaminoalkylamine.

11. Process according to claim 10, wherein the weight ratio of the fatty acid amidoalkyl dimethylamines, in particular fatty acid amidopropyl dimethylamines, provided in process step a) to the dimethylaminoalkylamines. in particular dimethylaminopropylamine, provided in process step a) is in the range from 99.7:0.3 to 90:10. preferably from 99.6:0.4 to 95:5, more preferably from 99.5:0.5 to 98:2.

12. Process according to claim 10, characterized in that process step b) is carried out at a temperature within a range from 60° C. to 120° C., more preferably 80° C. to 100° C.

13. Process according to claim 10 at least one of claims 10, characterized in that process step b) is carried out within a pH range from 7.5 to 10.5. preferably from 8.0 to 10.0. measured undiluted at 98° C.

14. A method of producing a cosmetic and/or dermatological formulation comprising providing the composition of claim 1 and at least one surfactant and mixing to form a cosmetic and/or dermatological formulation.
